(19) United States
(12) Patent Application Publication
(10) Pub. No.: US 2014/0170600 A1
(43) Pub. Date: Jun. 19, 2014

(54) METHODS AND SYSTEMS FOR MAKING COLORED DENTAL PARTS

(71) Applicant: Park Istre II, LLC, Dallas, TX (US)

(72) Inventor: Christopher Michael Koncewicz, Addison, TX (US)

(73) Assignee: Park Istre II, LLC, Dallas, TX (US)

(21) Appl. No.: 14/108,271

(22) Filed: Dec. 16, 2013

Related U.S. Application Data
(60) Provisional application No. 61/737,588, filed on Dec. 14, 2012.

(51) Int. Cl. A61C 13/08; A61C 13/00; A61K 6/02; A61C 5/10

(52) U.S. Cl. CPC A61C 13/082; A61C 5/10; A61C 13/0012; A61K 6/0255; A61C 13/0018
USPC 433/222.1; 433/223

(57) ABSTRACT

The invention provides a method for forming a colored dental part comprising forming an oxide layer on a metal base material, and submerging the metal base material in an electrolyte solution to bring to completion the formation of the oxide layer and forming the colored dental part.

### CROSS-REFERENCE TO RELATED APPLICATIONS

This application claims priority to, and incorporates by reference, for any purpose, the entire disclosure of, U.S. Provisional Patent Application No. 61/737,588, filed Dec. 14, 2012.

### TECHNICAL FIELD

The present invention relates to colored dental parts and methods for their preparation and more particularly, but not by way of limitation, to utilizing energy such as, for example, a laser, to create an oxide of a material, which oxide presents a color to an observer for a dental product.

### BACKGROUND

In the discussion that follows, reference is made to certain structures and/or methods. However, the following references should not be construed as an admission that these structures and/or methods constitute prior art. Applicants expressly reserve the right to demonstrate that such structures and/or methods do not qualify as prior art.

In the field of dentistry, many dental products can be engineered to mimic biological systems functionally, but often lack aesthetic appeal. Often times functional parameters may be sacrificed to ensure the component has visual appeal. A prime example of this is the use of porcelains, more generally ceramics, in dental crowns to mimic the visual appearance of human enamel. While porcelain has beneficial coloration and depth or opacity, it lacks the strength and ductility required to make chair-side modifications needed to fit each person's unique tooth structure. In addition, porcelain's brittle nature can lead to cracking and chipping during occlusal mastication or abrupt impact from a foreign material. Over the years, this has led to many combinations of materials to offer abrasion/wear resistance and ductility regionally, while not offering a complete solution for both functionality and aesthetic appeal. Similar compromises can often be made into products such as orthodontic brace wire, retainer wire, partial denture clasps, dental implants, abutments, and other dental products.

Occasionally, a complex new material or design is discovered that satisfies both the aforementioned functionality and aesthetic appeal, but is often uneconomical for a variety of reasons. Most importantly, the high cost often creates a barrier to widespread adoption of new medical technology.

What is needed is a simple and cost-effective system and method for the coloration of shaped dental parts with aesthetic appeal and the ability to mimic human tooth enamel while having the functional performance required in use.

### SUMMARY OF THE INVENTION

In broadest terms, the invention relates to utilizing energy such as, for example, a laser, to create an oxide of a material, which oxide presents a color to an observer for a dental product. The metal base material is of a type suitable for use in dental applications and may, in various embodiments, be coated with a ceramic. In other embodiments, the metal base material is uncoated. In accordance with one aspect of the present invention, a method for forming a colored dental part is provided. The method includes forming an oxide layer of a desired color on a metal base material, and submerging the metal base material in an electrolyte solution to complete formation of the oxide layer and forming the metal base material into a dental part.

In accordance with another aspect of the invention, a method for forming a colored dental part is provided. The method includes forming a ceramic layer on a metal base material of a type suitable for use in dental applications, forming an oxide layer of a desired coating on the ceramic coating, and submerging the ceramic-coated metal base material in an electrolyte solution to complete formation of the oxide layer and forming the forming the metal base material into a dental part.

In accordance with yet another aspect of the invention, a colored dental part is provided, the colored dental part includes a metal base material of a type suitable for use in dental applications and an oxide layer on the metal base material, wherein the oxide layer has a thickness of about 10 to 600 μm.

The above summary of the invention is not intended to represent each embodiment or every aspect of the present invention. Particular embodiments may include one, some, or none of the listed advantages.

### BRIEF DESCRIPTION OF THE FIGURES

A more complete understanding of the methods of the present invention may be obtained by reference to the following Detailed Description when taken in conjunction with the accompanying figures, wherein:

FIG. 1 is a flow diagram of a manufacturing process for the coloration of dental products according to an exemplary embodiment;

FIG. 2 is a cross-sectional diagram of a metal base material having an oxide layer applied thereto according to an exemplary embodiment; and

### DETAILED DESCRIPTION

In broadest terms, the invention relates to utilizing energy such as, for example, a laser, to create an oxide of a material, which oxide presents a color to an observer for a dental product. The coloration of dental products employs the thin film effect of one or more coating layers formed on a metal base material. This thin film has a visual characteristic in that a white light source reflects/refracts from a surface of the thin film at different wavelengths, seen to the human eye as differing colors. While having this visual characteristic, the thin film often improves on physical characteristics of the metal base material, including, without limitation, the hardness and abrasion resistance of the metal base material. One or more coating layers are largely dependent on the metal of the metal base material and the surrounding gaseous environment during molecular-surface excitations between the coating material and the metal itself. In some embodiments, the metal of the metal base material includes, without limitation stainless steel, stainless steel alloys, titanium alloys and zirconium alloys. The material of the one or more coating layers may include, without limitation, NiB, CrN, TiN, ZrN, DLC, TiCN, TiAlN, or AlTiN, TiO₂, ZnO, ZrO₂, ZrSiO₄, CaO, SiO₂, Al₂O₃, MgO, Y₂O₃, and Ce₂O₃.

Laser technology can provide commercially-available laser sources for producing thin film oxidation at relatively precise thicknesses, locations, and orientations on the metal base materials. Certain wavelengths ranging from 248 to 10,600 nm and of gas or solid-state constructions can be employed. Common laser gain media can include without limitation, Nd:YAG, YVO4, CO2, KrF, and Ar⁺ for use in the coloration of base material. A thickness and orientation of the thin film oxidation can play a large role in the color reflected and perceived by the human eye. The thickness and orientation of the thin film oxidation can both be a direct result of precise control and delivery of the stimulated emission of photons from the laser gain medium in conjunction with a galvonometer-type or gantry/bridge-type scan head.

In some embodiments, control of the laser can be by setting the laser parameters. The pulse width can be selected width in a range of from about 5 to about 1000 ns, and in some embodiments from about 7 to about 200 ns; however any range may be utilized. The frequency can be selected in a range of continuous emission to about 1.0 MHz and in some embodiments from about 20 to about 500 ns; however any range may be utilized. The pulse energy per area can be selected in the range of from about 0.1 to about 10 J/cm², and in some embodiments from about 0.8 to about 3.2 J/cm²; however any range may be utilized. The deviation from focal plane can be selected in the range of from about −85 to about 85 μm, and in some embodiments, from about −25 to about 25 μm; however any range may be utilized. The focal spot diameter can be selected in the range of from about 5 to about 300 μm, and in some embodiments from about 50 to about 120 μm; however any range may be utilized. The marking speed can be selected in the range of from about 5 to about 30,000 mm/s, and in some embodiments, from about 20 to about 5000 mm/s; however any range may be utilized. The line spacing can be selected in the range of from about 1 to about 300 μm, and in some embodiments from about 5 to about 150 μm; however any range may be utilized.

To create an accurate and repeatable formation of the coating layer on the surface of the metal base material, the gaseous environment can be adjusted during application of the coating layer. Atmospheric gases and temperature fluctuations can be manipulated to alter the formation method and resulting appearance. In connection with the gases surrounding the laser, certain concentrations of atmospheric gases can be increased to accelerate or decelerate oxide growth in the coating layer. In particular, increased oxygen levels accelerate oxide growth while deceleration can be achieved by displacing oxygen with gases such as argon, or by pulling a vacuum. The gas-to-metal kinetics plays an important role during oxide formation. The rate at which the gas molecules strike the solid surface during heating and solidification can determine whether the molecules will be absorbed or rebounded resulting in varied outcomes, including surface strength and appearance. Particularly, a linear flow of oxygen for oxide growth across the surface of the base material can be selected in the range from about 0.001 to about 0.023, m³/s, and in some embodiments, from about 0.003 to about 0.007 m³/s; however any range may be utilized.

Temperature control can play an important role in the thermodynamic state of the material and resulting growth of the oxide layer of the coating material. In general, an oxide becomes less thermodynamically stable with increasing temperature. The differential temperature of the locally-heated zone resulting from laser radiation with respect to the ambient temperature of the material can effect oxide growth. Laser-pulse energy and scanning velocity can be manipulated to vary energy imparted on the local zone. To elevate the temperature of the metal base material, energy can be imposed through radiation (via, for example, infrared lights), conduction (via, for example, a resistive heating element), or convection (via, for example, blowing heated gases over surface) in the range of about 75 to about 450° C., and in some embodiments of about 260 to about 345° C.; however any range may be utilized. Conversely, to lower the temperature, the same modes of heat transfer can be utilized through intimate contact with finned heat sinks and cooler gases, or refrigerated systems in the range of about −40 to about 50° C., and in some embodiments of about −5 to about 8° C.; however any range may be utilized. After pre-heating or cooling and localized heating via laser radiation, the rate of solidification of the local area additionally plays an important role. Rapid solidification can be achieved by imparting a large rate of heat transfer immediately after irradiation. Common methods of cooling include, but are not limited to, rapid submersion in an electrolyte bath or intimate contact with a material of high thermal conductivity, both of substantially lower ambient temperatures.

In some embodiments, a surface roughness of the metal base material can be an important factor. For example, the smoother the finish of the metal base material, the higher a binding energy and a larger range of contrasting colors can be achievable. Additionally, a polarity of the metal base material can play a role in effective oxide thickness and orientation. The metal base material can be polarized using ferromagnetic polarization such as, for example, electrically stimulated polarization and magnetically-induced polarization. In addition to the metal base material, a coating layer can offer better coloration and depth or enhanced durability (wear/abrasion resistance). The coating layers can be of desirable hardness and ductility, while maintaining biocompatibility and corrosion resistance. Excessive nickel content may be of concern as well because of the possibility of nickel allergies found in children.

The coating material can be applied to the surface of the metal base material by utilizing heat generated by fusing the coating material to the metal base material. The coating material can be selected based on its ideal coloration and physical properties. An important factor affecting the application of the coating material is the surface roughness of the metal base material. An increased surface area of the metal base material through micro-scale roughness can enable an increased bonding strength between the metal base material and the coating material. Examples of such coating materials include, without limitation, metal oxide ceramics such as, for example, CaO₃SiZr, MgO₃SiZr, TiO₂, ZnO, ZrO₂, ZrSiO₄, Al₂O₃, SiO₂, MgO, Y₂O₃, CeO₂, Ce₂O₃, Fe₂O₃, Er₂O₃, MnO₂, Pr₂O₃, Pr₆O₁₁, Bi₂O₃, CaO, Tb₂O₃, and Cr₂O₃ or any combination thereof. In some embodiments, binders or lubricants, can be used. The use of such coating material may, as a general rule, be uneconomical in temporary dental applications. However, given that the thickness of the coating material can be relatively small in comparison to the metal base material, the colored dental part can exhibit the positive qualities of the coating material while the manufacturing of the colored dental part can remain cost effective.

In some embodiments, a coating material such as, for example, a metal powder can be used. A thin-layer coating can be obtained, which thin-layer coating can require densification prior to sintering in order to reduce the porosity and ultimately achieve full bonding and mechanical performance. This densification can be achieved by mechanical or hydraulic compaction, using methods such as, for example, hot or cold isostatic pressing, single-axis die pressing, and multi-axis die pressing. To create a consistent thickness of the coating material, the metal base material can be electrically charged to attract the oppositely-charged ions in an aerated environment prior to compaction. Aeration of the environment can be accomplished by suspending the metal powder in a liquid or gas network flowing over the base material substrate. Compressed air can be mixed with the metal powder through staged nozzles prior to entering the work area surrounding the metal base material. Alternatively, the coating of the metal powder can be weighed and poured into a cavity around the metal base material. The metal base material can be suspended in the middle of the tooling cavity while the metal powder can be uniformly distributed by means of spinning/ centrifugal force or vibratory oscillations.

Following densification, the particles of the coating material can be sintered into an amorphous state to reduce porosity and create an intimate bond between the metal base material and the coating material. Sintering can be achieved by means of radiation from the laser source, or via radiation, convection, and/or conduction from a standard sintering furnace, inductive, or microwave heating source. If sintering is achieved by means other than the laser source, the laser can be used in a secondary operation to create the coating layer of metal oxide which metal oxide layer can be formed from the coating material and surrounding gaseous environment.

A ceramic coating layer can be formed on the metal base material prior to laser coloration to allow the use of relatively small amounts of higher grade coatings in proportion to the metal base material to which the ceramic coating can be bonded. The ceramic coating layer can include, without limitation, electroless nickel optionally reinforced with, for example, diamond, silicon carbide, boron nitride, or polytetraflouroethylene (“PTFE”) particles. The ceramic coating layer can also include, without limitation, NiB, CrN, TiN, ZrN, TiCN, TiAlN, or AlTiN coatings which coatings can be applied through, for example, chemical-vapor deposition, sputtering, and/or thermal spray. Some of the material in the ceramic coating layer may include ceramics, however the dental part can lack in ductility, which lack of ductility can lead to failures in flexural applications. To address this issue, regional application of the ceramic coating layer on the metal base material can have the benefit of wear/abrasion resistance, where needed, and ductility in flexing elsewhere where optimal coloration and abrasion resistance may not be required. Other coating methods include, without limitation, the passivation of stainless steel to remove exogenous iron or iron compounds from the surface, and anodizing techniques with or without PTFE additives.

The formation of the coating material on the metal base material prior to sintering can be applied through a slurry in which slurry an evaporative media can be combined. The slurry of foreign material and evaporative media can be applied by, for example, spraying, brushing, or submerging. Once evenly applied to the metal base material, the coating material can then be heated by, for example, laser radiation to create a strongly-bonded coating.

In some embodiments, additional coatings may be applied on the ceramic and oxide layer. These additional coatings can have advantages such as, for example, providing a protective barrier over the ceramic and/or oxide layers, contributing to adding service life to dental parts, and adding depth/opacity to the physical appearance. Commercially-available transparent or translucent ceramic coatings can offer this protection as well as transparent or translucent ultraviolet cured acrylates, epoxies, and other various common dental resin-based composites.

In some embodiments, for a dental part which can be manufactured in a metal stamping method, a two-dimensional method of laser application of a metal oxide layer on the metal base material can be utilized. The metal base material coil may be colored prior to entering a progressive die press or turret press. One advantage is that the micro-porosity in the metal oxide layer can allow for a flexural modulus similar to the metal base material. This in turn allows the dental part to be formed from a flat sheet metal coil, to a metal oxide coating flat sheet metal coil, and ultimately a three-dimensional metal oxide coated component. This two-dimensional laser metal oxide application can be accomplished by utilizing commercially-available laser sources in combination with a galvanometer-based or gantry-bridge type scan head. Most commonly, the galvanometer-based scan head can be utilized due to higher reliability and marking speed in production and direct radiation output by the laser gain medium through a beam expanding collimator into the scan head. The scan head can then direct the beam two dimensionally onto the metal base material by means of minors rotated by galvanometers through F-theta or Telecentric lensing.

Optionally, a beam homogenizer can be used to more evenly distribute energy of the laser across a spot diameter, commonly referred to as a “Top-Hat” mode in comparison to the typical Gaussian distribution. This can allow for the metal base material coil to be marked in batches before entering the presses, or continuously as the material can be fed utilizing positional feedback via an encoder. Many marking patterns can be possible, with the most common being, for example, a linearly-stepped pattern in one dimension, a cross hatched pattern in two dimensions, a spiral pattern in two dimensions radially, and a spot filled pattern consisting of a series of dots. Out of the patterns mentioned, the spot pattern yields the least directionally-dependent color based on incident viewing angle.

A three-dimensional method of application can be used to color a formed metal base material. In such embodiments, once the metal base material is formed into its final shape, laser application of the coating layer can be achieved in several different methods. In an exemplary embodiment of a dental crown, the geometric shape can present challenges for the industry standard three-dimensional laser setup. In looking at any arbitrary cross-section of a crown, the outer surface can extend past about −40 to about 40 degrees angularly to the perpendicular axis to the laser source. In essence, no one orientation of the crown can allow for complete application of the coating layer to surface. In such an instance, either the dental part or the laser source can be manipulated to apply the coating on the totality of the outer surface. In embodiments where the laser scan head remains stationary, the dental part can be positioned in different orientations, and soft automation, such as robotics, or hard automation in a batch type or continuous methodology can be used. Common commercially-available industrial robotics can be used in four, five, or six axis articulated robotics custom tailored with end effectors to hold the dental part in an accurate and repeatable fashion with respect to the laser source in the range of about −0.1 to 0.1 mm, and in some embodiments of about −0.02 mm to 0.02 mm.

In some embodiments, another method can include the use of the custom design of any combination of translational and rotary axes. These can be referred to as hard automation, to achieve the multiple orientations. In this batch-type method, once the dental product is fixed in space, the laser can be programmed with a three-dimensional model of the surface of the metal base material to be marked via, for example, CAD technology. The laser can then pass over the surface of the metal base material in a pattern, typically linear, which can be manipulated by adjusting the moving lens with respect to the focusing lens in concert with the x and y-axis galvanometers found in the scan head.

In some embodiments, another method can include continuous marking of the outer surface of the dental part in which the automated positioning of the dental part can be moved in conjunction with the laser scan head to allow one marking pattern over the entire surface of the base material. The robot or hard automation can be controlled by the laser system, or vice versa, via master/slave configuration. The pattern in which the laser can cover the colored area can be varied depending on the geometry of the dental part, for instance linearly back and forth, spiral, and/or topographically.

In some embodiments, another option can be available to mark the surface of the metal base material in different orientations in which options the product can remain stationary and the laser scan head can be mounted on the end of a robotic arm or hard automation actuator(s). A conveyor approach can be used and each dental product can be rapidly fed through the cell while the sensitivity of the galvanometers in the scan head can be safeguarded while monitoring the acceleration. This option can be applied to either a batch or continuous configuration.

In some embodiments, continuous marking can be applied as an alternative to batch marking due to the consistency of the oxide growth in the coating layer. In batch marking, each discrete region marked can have a perimeter that can be difficult to align with the previously marked region. This commonly leads to patches of colored areas separated by visible lines.

In some embodiments, a design of coloration and markings can be added to any dental part with many options of appearances that can be created on the surface of the dental part while maintaining the coloration and depth/opacity of the dental part. A near white/off-white can be achieved to mimic human enamel and dentin (dentin tooth structure) with a small product identifier such as model number, batch number, or even serial number shown in digits, barcode, or two-dimensional barcode. The product identifier can be used to store a unique patient identifier to which historical records can be linked in a remote database referencing the product identifier. An advantage of product identification can be, for example, in assisting medical staff in referencing maintenance on the medical device should original records be lost, and assisting law enforcement agencies in referencing patient records that can be of utility in forensic analysis. Another advantage can be that pictorial art or brands can be projected on any surface of the product to have a unique design shown. Coloration can be varied to create a multi-color image gearing to the patient category, for example an image of child's favorite superhero or a logo of an adult's beloved football team.

The methods of the invention can include the coloration of orthodontic brace wire. Similar to dental crowns, the intended use of orthodontic brace wire can create difficult physical performance criteria, that need addressing, generally at the expense of aesthetics. Common brace wire materials such as nickel titanium and stainless steel, have a silver metallic appearance creating a large contrast to the adjacent white/off-white teeth. Coatings such as PTFE can be used, with a disadvantage that the flexural demand on the wire in aligning teeth often cracks and breaks the coating revealing the surface of the base material. Oxide growth using laser heating and forming may offer visual appeal through camouflage while maintaining the durability of the orthodontic brace wire while in use.

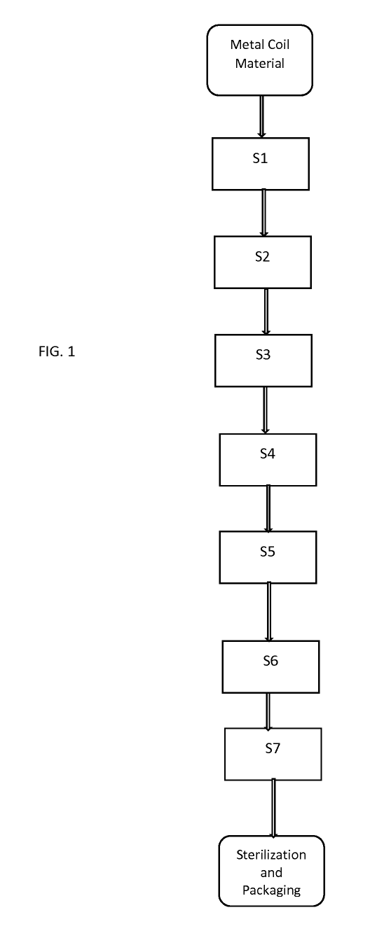

FIG. 1 is a flow diagram of a manufacturing process for the coloration of dental products.

In step S1, the metal base material can be heated depending on the dental part. In some embodiments, heating can be used to clean a substrate of contaminants which contaminants may have been introduced in prior processing, handling, or shipment. Additionally, heating can be useful in tempering the base material in order to relieve stress concentrations and create homogeneous surface conditions. Inert gasses including argon and nitrogen can be used in conjunction with vacuum and can be proven effective at flow rates in the range of about 1 to about 25 L/min, and in some embodiments, of about 15 to about 18 L/min to displace the oxygen; however any range may be utilized. Sufficient heating in the range of about 75 to about 450° C., and in some embodiments about 260 to about 345° C. and for about 2 to about 60 minutes, and in some embodiments, for about 15 to about 20 minutes; however any range may be utilized.

In step S2, the metal base material can be cleaned further of contaminants and surface oxides. In some embodiments, chemical cleaning can be used by submerging the base material in an alkaline or acidic ultrasonic bath in a temperature range of about 37 to about 70° C., and in some embodiments of about 55 to about 60° C., for about 2 to about 45 minutes, and in some embodiments for about 5 to about 15 minutes; however any range may be utilized. In some embodiments, the top surface layer can be mechanically removed with abrasive media to produce a surface with a roughness of about Ra 0.5 μm. In some embodiments, to ensure complete surface preparation, chemical cleaning and mechanical removal can be used in tandem.

In step S3, an optional ceramic coating layer can be applied to the base metal material to enhance performance characteristics such as, for example, hardness, abrasion resistance, strength, biocompatibility, and coloring. In some embodiments, the ceramic coating layer can include electro-less nickel optionally reinforced with, for example, diamond, silicon carbide, boron nitride, or PTFE particles. In other embodiments, the ceramic coating layer can also include, for example, NiB, CrN, TiN, ZrN, DLC, TiCN, TiAlN, or AlTiN, TiO₂ , ZnO, ZrO₂, ZrSiO₄, CaO, SiO₂, Al₂O₃, MgO, Y₂O₃, and Ce₂O₃, that can be applied by, for example, vapor deposition, sputtering, and/or thermal spray and can provide good wear properties and attractive cosmetics following laser processing.

In some embodiments, harder coatings such as ceramics may exhibit poor ductility, which may potentially lead to a failure in flexural applications, therefore the application of pre-coating on limited portions of the product can have the advantage of improved wear/abrasion resistance where needed, and ductility in flexing where optimal coloration and abrasion resistance may not be required. Other methods can include passivation of stainless steel to remove exogenous iron or iron compounds from the surface, and anodizing techniques with or without PTFE additives. Additionally, the ceramic coating layer can include, for example, powdered metal oxide ceramics such as CaO₃SiZr, MgO₃SiZr, TiO₂, ZnO, ZrO₂, ZrSiO₄, Al₂O₃, SiO₂, MgO, Y₂O₃, CeO₂, Ce₂O₃, Fe₂O₃, Er₂O₃, MnO₂, Pr₂O₃, Pr₆O₁₁, Bi₂O₃, CaO, Tb₂O₃, and Cr₂O₃ or any combination thereof which can be attached and sintered to the metal base material. Method aids such as binders or lubricants can be used to improve processing capabilities. The thickness of the ceramic coating layer is in the range of about 1 μm to about 200 μm, and in some embodiments of about 50 to about 80 μm; however any range may be utilized. In some embodiments, the ceramic coating layer may require densification prior to sintering in order to reduce porosity and ultimately achieve full bonding and mechanical performance. Densification can be achieved by mechanical or hydraulic compaction, using methods such as hot or cold isostatic pressing, single-axis die pressing, and multi-axis die pressing in the range of about 1000 to about 2760 bar, and in some embodiments of about 1800 to about 2200 bar to create a pressed form commonly referred to as the green compact. To create a consistent ceramic coating layer thickness, a negative charge can be placed on the base material to attract the positively charged ions in an aerated environment prior to compaction. Aeration of the environment can be accomplished by suspending the metal powder in a liquid or gas network flowing over the base material substrate. Compressed air can be mixed with the material of the ceramic coating layer through staged nozzles prior to entering the work area surrounding the base material substrate. Other methods utilize a solvent-based aerosol suspension to spray, brush, or submerge the material of the ceramic coating layer on the surface. Alternatively, the material of the ceramic coating layer can be weighed then poured into a cavity around the base material prior to compaction. The metal base material can be suspended in the middle of the tooling cavity while the material of the ceramic coating layer can be uniformly distributed around by means of spinning/centrifugal force or vibratory oscillations.

In step S4, the metal base material can be prepared for application of an oxide layer for laser heating. The metal base material can be prepared for laser heating through intimate contact with a heat sink in addition to preparing the proper surrounding gaseous environment. In some embodiments, when the cross-sectional thickness of the metal base material is in the range of up to about 0.9 mm, mechanical support through vacuum pressure to a heat sink fixture possessing adequate thermal conductivity of at least about 150 W/m-K may be required. In some embodiments, the roughness of the contacting surface of the heat sink fixture can be less than about 0.1 μm Ra. The heat sink fixture can be cooled by means of a heat exchanger, which heat exchanger can be sized to match the heat output by the laser source in step S6. A metal base material cross-sectional thickness larger than about 0.9 mm typically has sufficient heat capacities and can resist warping in heating, thus reducing the need for a heat sink fixture. In some embodiments, to ensure a consistent surrounding gaseous environment, the flow of oxygen across the surface of the base material can be in the range of about 0.001 to about 0.023 m³/s, and in some embodiments of about 0.003 to about 0.007 m³/s; however any range may be utilized.

In step S5, to the metal base material with or without a ceramic coating layer, a oxide layer can be applied by heating the metal base material with radiation from a laser source. Certain wavelengths in the range of about 248 to about 10,600 μm and of gas or solid-state constructions can be employed. Common laser gain media can include without limitation Nd:YAG, YVO4, CO2, KrF, and Ar⁺. Precise control and delivery of the stimulated emission of photons from the laser gain medium in conjunction with a galvonometer-type or gantry/bridge-type scan head can deliver a high speed local region of heat on the base material. In specific embodiments, control can be achieved by setting the laser parameters. The pulse width can be selected width in a range of from about 5 to about 1000 ns, and in other embodiments from about 7 to about 200 ns; however any range may be utilized. The frequency can be selected in a range of continuous emission to about 1.0 MHz and in other embodiments from about 20 to about 500 ns; however any range may be utilized. The pulse energy per area can be selected in the range of from about 0.1 to about 10 J/cm², and in other embodiments from about 0.8 to about 3.2 J/cm²; however any range may be utilized. The deviation from the focal plane can be selected in the range of from about −85 to about 85 μm, and in other embodiments, from −25 to about 25 μm; however any range may be utilized. The focal spot diameter can be selected in the range of from about 5 to 300 μm, and in other embodiments from about 50 to about 120 μm; however any range may be utilized. The marking speed can be selected in the range of from about 5 to 30,000 mm/s, and in other embodiments, from about 20 to about 5000 mm/s; however any range may be utilized. The line spacing can be selected in the range of from about 1 to about 300 μm, and in other embodiments from about 5 to about 150 μm; however any range may be utilized. Specific combinations and control of these parameters can yield unique and consistent coloration of the oxide layer. In some embodiments, the oxide layer can include without limitation, compounds such as, for example, CaO3SiZr, MgO3SiZr, TiO2, ZnO, ZrO2, ZrSiO4, Al2O3, SiO2, MgO, Y2O3, CeO2, Ce2O3, Fe2O3, Er2O3, MnO2, Pr2O3, Pr6O11, Bi2O3, CaO, Tb2O3, and Cr2O3 or any combination. The thickness of the oxide layer can be in the range of about 10 to about 600 nm, and in some embodiments of about 60 to about 360 μm thick; however any range may be utilized.

In step S6, the metal base material can be rapidly cooled by pouring or submersion in an electrolyte solution to bring to completion the formation of the oxide layer in a controlled manner and allow partial infiltration of the elements contained in the electrolyte solution into the oxide layer. The electrolyte solution can be acidic or alkaline and can include without limitation one or more of Na₂SiO₃, KOH, KF, NaAlO₂, NaOH, SiC, NaPO₂H₂.H₂O, K₂Al₂O₄, Na₃PO₄, Na₂CO₃, Zr(OH)₂CO₃.ZrO₂, and EDTA-2Na in any combination, with distilled water.

In step S7, the oxide layer on the metal base material can be polished to a smooth surface roughness. The smooth surface roughness can be a factor in ensuring that biological contaminants do not adhere to the dental part during its service life. Various polishing methods can be used, including without limitation, vibratory or rotary tumbling of the dental part adjacent to abrasive or burnishing media in the range of about 70% media/30% dental part to about 99% media/1% dental part with or without water; however any range may be utilized. Additionally, pneumatically-assisted blasting media can be utilized including without limitation, glass beads, crushed glass, alumina, silicon carbide, plastic abrasive, coal slag, pumice, steel shot, steel grit, corn cob, and walnut shells. Often times multiple iterations of polishing can be required in which the media roughness can be decreased progressively. In some embodiments, the surface roughness of the polished dental part can be in the range of about Ra 0.10 μm or less.

In step S8, the metal base material of step S7 can be coated with a dental composite to protect from any potential wear-related abrasion while adding depth or opacity to the appearance. Dental composites suitable for coating include, without limitation, commercially available transparent or tinted poly-ceramic coatings, transparent or tinted ultraviolet-cured acrylates and epoxies and other various common dental composites. In some embodiments, poly-ceramic compounds can be formulated of both polymeric and ceramic components including without limitation, SiO₂ and C₇H₄ClF₃ formulated with additional catalysts. In some embodiments, commercially available hydrophobic per-fluorinated silanes can be used. The coating precursor can be solvent-based and can be applied by means of spraying, brushing, or submerging. In some embodiments where spraying is used, opposing electrical charges between the precursor and the dental part can enhance the consistency of distribution of the coating. The coating can be activated by means of energy influx via sintering or ultraviolet radiation, or curing in ambient conditions. In the case of sintering, heating can be in the range of about 50 to about 200° C., and in some embodiments, of about 65 to about 180° C., and the coating thickness can be in the range of about 1 to 60 μm, and in some embodiments of about 10 to about 28 μm; however any range may be utilized.

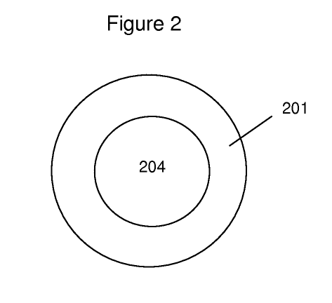

FIG. 2 is a cross-sectional diagram of a metal base material having an oxide layer applied thereto. An oxide layer 201 can be applied to a metal base material 204 by applying energy to the metal base material 204 via, for example, radiation from a laser source. In a typical embodiment, the laser source is operated according to the parameters discussed above with respect to step S5. By way of example, the metal base material 204 is shown in FIG. 2 is a wire of the type used, for example, in orthodontics. The metal base material 204 may be, for example, a crown, an implant, a veneer, an orthodontic wire, or other dental apparatus. In other embodiments, the metal base material 204 may be a length of raw material such as, for example, coil stock, to which raw material the oxide layer 201 is applied. In a typical embodiment, the oxide layer 201 is formed on the entirety of the metal base material 204. In other embodiments, the oxide layer 201 is not formed on the entirety of the metal base material 204 but, rather, is formed on any portion of the metal base material 204.

Although various embodiments of the method and apparatus of the present invention have been illustrated in the accompanying figures and described in the Detailed Description, it will be understood that the invention is not limited to the embodiments disclosed, but is capable of numerous rearrangements, modifications, and substitutions without departing from the spirit and scope of the invention.

What is claimed is:

1. A method for forming a colored dental part, the method comprising:
forming an oxide layer on a metal base material, the metal base material being of a type suitable for use in dental applications;
submerging the metal base material in an electrolyte solution to complete formation of the oxide layer; and
forming the metal base material into a colored dental part.

2. The method of claim 1, wherein the forming the oxide layer comprises:
heating the metal base material with a laser; and flowing an oxygen-enriched gas across the metal base material.

3. The method of claim 2, wherein the laser is at a wavelength in the range of about 248 to about 10,600 μm.

4. The method of claim 2, wherein a flow of the oxygen-enriched gas across a surface of the metal base material is about 0.001 to about 0.023 m³/s.

5. The method of claim 1, comprising polishing the oxide layer to a surface roughness of about Ra of 0.1 μm or less.

6. The method of claim 1, comprising coating the oxide layer with a dental composite.

7. The method of claim 1, wherein a thickness of the oxide layer is about 10 to about 600 μm.

8. The method of claim 1, wherein the metal base material comprises at least one of stainless steel, stainless steel alloys, titanium alloys and zirconium alloys; and
the oxide layer comprises at least one of CaO₃SiZr, MgO₃SiZr, TiO₂, ZnO, ZrO₂, ZrSiO₄, Al₂O₃, SiO₂, MgO, Y₂O₃, CeO₂, Ce₂O₃, Fe₂O₃, Er₂O₃, MnO₂, Pr₂O₃, Pr₆O₁₁, Bi₂O₃, CaO, Tb₂O₃, and Cr₂O₃.

9. The product formed by the method of claim 1.

10. A method for forming a colored dental part, the method comprising:
applying a ceramic coating to at least a portion of a metal base material, the metal base material being of a type suitable for use in dental applications;
applying energy to the ceramic coating and the metal base material to create an oxide layer thereon;
submerging the metal base material in an electrolyte solution to complete creation of the oxide layer; and
forming the metal base material into a colored dental part.

11. The method of claim 10, wherein the forming the ceramic coating is by at least one of chemical vapor deposition, sputtering, thermal spray, and powder compaction of one or more compounds selected from NiB, CrN, TiN, ZrN, DLC, TiCN, TiAlN, AlTiN, TiO₂ , ZnO, ZrO₂, ZrSiO₄, CaO, SiO₂, Al₂O₃, MgO, Y₂O₃, and Ce₂O₃.

12. The method of claim 10, wherein the applying energy comprises:
heating the metal base material with a laser; and
flowing an oxygen-enriched gas across the metal base material.

13. The method of claim 12, wherein the laser source is at a wavelength in the range of about 248 to about 10,600 μm.

14. The method of claim 12, wherein a flow of the oxygen-enriched gas across the surface of the metal base material is about 0.001 to about 0.023 m³/s.

15. The method of claim 10, comprising polishing the oxide layer to a surface roughness of about Ra of 0.1 μm or less.

16. The method of claim 10, comprising coating the oxide layer with a dental composite.

17. The method of claim 10, wherein a thickness of the oxide layer is about 10 to about 600 μm.

18. The method of claim 10, wherein the metal base material comprises at least one of stainless steel, stainless steel alloys, titanium alloys and zirconium alloys; and
the metal oxide comprises one or more of CaO₃SiZr, MgO₃SiZr, TiO₂, ZnO, ZrO₂, ZrSiO₄, Al₂O₃, SiO₂, MgO, Y₂O₃, CeO₂, Ce₂O₃, Fe₂O₃, Er₂O₃, MnO₂, Pr₂O₃, Pr₆O₁₁, Bi₂O₃, CaO, Tb₂O₃, and Cr₂O₃.

19. The product formed by the method of claim 10.

20. A colored dental part comprising:
a metal base material of a type suitable for use in dental applications;
an oxide layer of a desired color formed on the metal base material; and
wherein the oxide layer has a thickness of about 10 to about 600 μm.

21. The colored dental part of claim 20, further comprising a ceramic coating layer applied to the metal base material.

22. The colored dental part of 20, wherein the metal base material comprises at least one of stainless steel, stainless steel alloys, titanium alloys, and zirconium alloys; and
the metal oxide comprises at least one of CaO₃SiZr, MgO₃SiZr, TiO₂, ZnO, ZrO₂, ZrSiO₄, Al₂O₃, SiO₂, MgO, Y₂O₃, CeO₂, Ce₂O₃, Fe₂O₃, Er₂O₃, MnO₂, Pr₂O₃, Pr₆O11, Bi₂O₃, CaO, Tb₂O₃, and Cr₂O₃.
